(19) United States
(12) Patent Application Publication
(10) Pub. No.: US 2005/0176650 A1
(43) Pub. Date: Aug. 11, 2005

(54) Stable parenteral formulation of levomepromazine and a method for stabilizing said formulation

(75) Inventor: Ralph A. Heasley, Union, KY (US)

Correspondence Address: WOOD, HERRON & EVANS, LLP, 2700 CAREW TOWER, 441 VINE STREET, CINCINNATI, OH, 45202, US

(73) Assignee: Xanodyne Pharmacal, Inc., Florence, KY (US)

(21) Appl. No.: 10/774,803

(22) Filed: Feb. 9, 2004

(51) Int. Cl. A61K 38/05; A61K 31/5415

(52) U.S. Cl. 514/18; 514/225.8

(57) ABSTRACT

The present invention relates to stabilized formulations for parenteral administration comprising a therapeutically effective amount of levomepromazine or a pharmaceutically acceptable salt thereof, and selected stabilizers in an amount effective to stabilize said formulation, to stable terminally sterilized levomepromazine formulations, to methods stabilizing pharmaceutical formulations comprising levomepromazine, and to methods of treating disorders using said formulation.

### BACKGROUND OF THE INVENTION

I. Field of the Invention

The present invention relates to a stabilized formulation for parenteral administration of levomepromazine. More particularly, this invention provides an injectable formulation of levomepromazine having an improved stability by using selected stabilizers.

II. Description of the Prior Art

Levomepromazine, [(−)-(2R)-3-(2-methoxy-10H-phenothiazin-10-yl)-N,N,2-trimethylpropan-1-amine], which is also commonly referred to as methotrimeprazine, is an active pharmaceutical ingredient possessing various therapeutic effects. For example, levomepromazine possesses analgegic, antiemetic, antipsychotic, tranquilizing, sedative, anxiolytic, antisialogogic, amnesic, and antihypertensive effects, and it is also a potent potentiator of anesthetics (analgesic adjuvant). More particularly, it enhances the effects of ether and hexobarbital anesthesia as well as morphine analgesia. It also exerts a potent anti-apomorphine effect, a hypothermic action three times more potent than that of chlorpromazine and strong antispasmodic and antihistaminic effects. Levomepromazine is capable of reversing epinephrine-induced hypertension, which has practically no effect against norepinephrine and acetylcholine. In rats, levomepromazine has been shown to readily protect against traumatic shock and produce deep local anesthesia following parasciatic injections.

Accordingly, levomepromazine is administered for many purposes. For example, it is administered to treat psychotic disturbances, including acute and chronic schizophrenias, senile psychoses, manic-depressive syndromes, and conditions associated with anxiety and tension, such as autonomic disturbances, personality disturbances, emotional troubles secondary to such physical conditions such as resistant pruritis, and the like. As an analgesic, it is administered to treat or alleviate pain due to cancer, zoster, trigeminal neuralgia, and intercostal neuralgia, as well as for phantom limb pains and muscular discomforts. As an analgesic adjuvant, it is administered as both a pre- and postoperative sedative and analgesic. As an anti-emetic, it is useful for the treatment of nausea and vomiting of central origin. As a strong sedative, it is useful for the management of insomnia.

While levomepromazine has been orally administered, it has been found to be particularly effective when parenterally administered, for example, via intramuscular injection, intravenous injection or continuous subcutaneous infusion.

Levoprome®, which previously was marketed in the United States but subsequently was withdrawn, was a formulation for injection of levomepromazine. It has been reported that Levoprome® contained ethylenediaminetetraacetic acid (EDTA), sodium chloride, sodium metabisulfite, citric acid, sodium citrate, benzyl alcohol, hydrochloric acid, and levomepromazine free base formulated at 20 mg/ml in a 10 ml multidose vial. Currently available parenterally administrable formulations of levomepromazine are found outside of the United States, which include Nozinan® and Levotomin®. These formulations contain 25 mg/ml or 20 mg/ml of levomepromazine. Nozinan® is reported to comprise ascorbic acid, sodium chloride, sodium sulfite, and water, in addition to levomepromazine, in the form of ampoules and vials.

These formulations for parenteral administration of levomepromazine, however, have been reported to have various disadvantages or drawbacks, especially in terms of meeting stability requirements. Levomepromazine has been generally reported to be sensitive to light and oxygen. For example, levomepromazine undergoes oxidation in the presence of oxygen to form multiple degradation products. Oxidation of the sulfur in the levomepromazine compound produces the two major decomposition products, levomepromazine sulfoxide and levomepromazine sulfone. N-desmethyl levomepromazine is a process impurity, a potential degradation product, and a metabolite of levomepromazine, which has been identified and characterized. Levomepromazine also degrades to other degradation products, which have yet to be isolated, identified and characterized.

In addition, parenteral levomepromazine formulations typically change colors over time. Such discoloration generally indicates a lack of stability and/or safety of the formulation. While a relatively impurity-free formulation is clear and colorless or almost clear and colorless, slight yellow is an acceptable color for a parenteral levomepromazine formulation. However, objectionable colors, such as dark yellow and salmon pink, which easily proceed over time to brown, have been reported with many parenteral levomepromazine formulations that have been exposed to oxygen and heat. These color changes are generally the result of the degradation of levomepromazine and/or other components in the formulation.

Current practices to control pharmaceutical formulations call for specifications on the concentration of the active ingredients as well as their decomposition products. Particularly, all of the known decomposition products must be reported, identified, or qualified in order for the formulation to gain government approval. If the levels of the decomposition products exceed the amounts established by the International Conference on Harmonisation in the Harmonised Tripartite Guideline—Impurities in New Drug Products Q3B(R) these amounts of decomposition products in the formulation must be qualified to meet regulatory requirements for product approval by the United States Food and Drug Administration (FDA). In addition to such regulatory-related complications, high levels of decomposition products generally result in considerable cost and time in their identification, monitoring and quantification. Furthermore, to comply with the government-regulated lower limits for decomposition products, lower storage temperatures and other costly manufacturing and/or packaging and/or storage measures are typically required. Such added measures add cost and time to the manufacturing process leading to higher costs and inefficiencies in the manufacture and commercialization of the final, parenterally administrable formulation. In addition, any unexpected toxicity found to be associated with any particular decomposition product may further result in a very low specification limit for approval, which generally shortens the formulation's shelf life.

Various attempts, therefore, have been made to improve stability of a formulation for parenteral administration of levomepromazine. For example, stabilizers have been used in a formulation for parenteral administration of levomepromazine to prevent oxidation of levomepromazine and/or color change. In particular, it is reported that, as stabilizers, Nozinan® contains sodium sulfite and ascorbic acid, and that Levoprome® contained EDTA and sodium metabisulfite.

It has been recognized that terminal sterilization is a preferred way to obtain sterility assurance. However, it was found that sulfite compounds, such as sodium metabisulfite or sodium sulfite, when used in levomepromazine formulations as a stabilizer actually are not stable after terminal sterilization, especially a sterilization procedure involving heat, such as autoclaving. Nozinan® and Levoprome®, therefore, have been manufactured using an aseptic technique. The aseptic technique used for Nozinan® and Levoprome® has been reported to assure a sterility of approximately 103 (the probability of a vial or ampoule being contaminated with a viable microorganism is one in one thousand). Such a level of sterility is, however, far less than the sterility that can be assured by terminal sterilization, which is about 106 to about 1012 or greater (the probability of a vial or ampule being contaminated with a viable microorganism is one in one million or one raised to the twelfth power). Therefore, it has been desired to produce a levomepromazine formulation that is capable of withstanding terminal sterilization while maintaining its physical and chemical stability.

Accordingly, there has been a significant need to develop a formulation for parenteral administration of levomepromazine having an improved stability, thereby preventing or reducing the formation and accumulation of degradation products in the formulation or discoloration of the formulation and assuring a longer shelf life. There is a further need to provide a levomepromazine formulation having a greater assurance of sterility than previous formulations by making it more resistant to terminal sterilization, especially to autoclaving and other sterilization procedures involving heat. There is a further need to provide these formulations more effectively, at less cost, and in a more convenient manner with respect to administration, than previous formulations.

### SUMMARY OF THE INVENTION

It now has been found that a formulation for parenteral administration of levomepromazine can be prepared with selected stabilizers to have an improved stability thereby effectively preventing or reducing the formation and accumulation of degradation products or discoloration resulting from oxidation and/or autooxidation of levomepromazine or other components in the formulation. Thus, such formulations assure a longer shelf life. It also has been found that a levomepromazine formulation comprising selected stabilizers can maintain its stability after terminal sterilization, such as autoclaving and other sterilization procedures involving heat, which assures a higher sterility than that obtained by an aseptic technic, and often may be required for a regulatory approval. In particular, selected stabilizers that are substantially free from sulfite compounds will permit terminal sterilization processes assuring a high sterility without sacrificing the desired stability. It further has been discovered that selected stabilizers can impart stability to the formulation less dependent on or without having to rely on the elimination of oxygen from the formulation.

Therefore, the present invention provides a pharmaceutical composition for a formulation for parenteral administration of levomepromazine, which has an improved stability. In accordance with one aspect of the present invention, the formulation comprises,
(a) a therapeutically effective amount of levomepromazine or a pharmaceutically acceptable salt thereof,
(b) ethylenediaminetetraacetic acid (EDTA) or a pharmaceutically acceptable salt thereof as a first stabilizer, and
(c) monothioglycerol (MTG) or glutathione as a second stabilizer,
wherein said stabilizers are present in an amount effective to stabilize said formulation, and wherein said formulation is subjected to sparging.

Another aspect of the present invention provides a formulation comprising:
(a) a therapeutically effective amount of levomepromazine or a pharmaceutically acceptable salt thereof,
(b) ethylenediaminetetraacetic acid (EDTA) or a pharmaceutically acceptable salt thereof as a first stabilizer,
(c) monothioglycerol (MTG) or glutathione as a second stabilizer, and
(d) ascorbic acid or a pharmaceutically acceptable salt thereof as a third stabilizer,
wherein said stabilizers are present in an amount effective to stabilize said formulation. In one embodiment of the present invention, the formulation comprises a therapeutically effective amount of levomepromazine or a pharmaceutically acceptable salt thereof, EDTA, MTG and ascorbic acid. In a preferred embodiment, the formulation is substantially free from sulfite compounds. In a further preferred embodiment, the formulation of the present invention is terminally sterilized, especially by a sterilization procedure involving heat, such as autoclaving. In another preferred embodiment, the formulation contains a concentration of total impurities of less than about 3% by weight per volume of the formulation.

Yet another aspect of the present invention provides a formulation comprising:
(a) a therapeutically effective amount of levomepromazine or a pharmaceutically acceptable salt thereof,
(b) ethylenediaminetetraacetic acid (EDTA) or a pharmaceutically acceptable salt thereof as a first stabilizer,
(c) ethylgallate or cysteine as a second stabilizer, and
(d) ascorbic acid or a pharmaceutically acceptable salt thereof as a third stabilizer,
wherein said stabilizers are present in an amount effective to stabilize said formulation, and wherein said formulation is subjected to sparging.

Still another aspect of the present invention provides a stable terminally sterilized formulation comprising a therapeutically effective amount of levomepromazine or a pharmaceutically acceptable salt thereof, wherein said formulation contains a concentration of total impurities of less than about 3% by weight per volume of the formulation and is terminally sterilized.

A further aspect of the present invention provides a method for stabilizing a formulation of levomepromazine, comprising:
(a) combining a therapeutically effective amount of levomepromazine or a pharmaceutically acceptable salt thereof, and a stabilizing amount of a combination of stabilizers in a medium to form a formulation, and
(b) sparging said formulation with an oxygen-free inert gas,
wherein said combination of stabilizers comprises (1) EDTA or a pharmaceutically acceptable salt thereof as a first stabilizer and (2) monothioglycerol (MTG) or glutathione as a second stabilizer.

Another aspect of the present invention provides a method for stabilizing a formulation of levomepromazine, comprising:
combining a therapeutically effective amount of levomepromazine or a pharmaceutically acceptable salt thereof, and a stabilizing amount of a combination of stabilizers in a medium suitable for parenteral administration to form a formulation,
wherein said combination of stabilizers comprises (1) EDTA or a pharmaceutically acceptable salt thereof as a first stabilizer, (2) monothioglycerol (MTG) or glutathione as a second stabilizer, and (3) ascorbic acid or a pharmaceutically acceptable salt thereof as a third stabilizer. In one embodiment of the present invention, the method further comprises subjecting said formulation to terminal sterilization such as autoclaving or other sterilization procedure involving heat. In yet a further embodiment, the formulation can be sparged with a substantially oxygen-free inert gas, which includes without limitation, nitrogen, carbon dioxide, argon and/or helium, to reduce or eliminate oxygen from the solution.

Still another aspect of the present invention provides a method for stabilizing a formulation of levomepromazine, comprising:
(a) combining a therapeutically effective amount of levomepromazine or a pharmaceutically acceptable salt thereof and a stabilizing amount of a combination of stabilizers in a medium suitable for parenteral administration to form a formulation, and
(b) subjecting said formulation to sparging with an oxygen-free inert gas,
wherein said combination of stabilizers comprises (1) EDTA or a pharmaceutically acceptable salt thereof as a first stabilizer, (2) ethylgallate or cysteine as a second stabilizer, and (3) ascorbic acid or a pharmaceutically acceptable salt thereof as a third stabilizer.

The present invention further provides a method for treating a disorder in a patient in need thereof, comprising administering to said patient an effective amount of a formulation of the present invention, wherein said disorder comprises psychosis, agitation, pain, migraine headache, nausea, vomiting, itching, hypertension, benign prostatic hypertrophy, excess gastrointestinal (GI) secretions, or sleeplessness.

These and other advantages and benefits of the present invention will be further appreciated in light of the detailed description of exemplary embodiments below.

### DETAILED DESCRIPTION OF EXEMPLARY EMBODIMENTS

The invention will be further appreciated in light of the following definitions:

The term “parenteral,” as used herein with respect to administering the formulation, is intended to generally refer to all methods of administering the formulation to a patient by a mode other than oral administration. Thus, parenteral administration includes administration by injection through other routes such as subcutaneous, intramuscular, intravenous, intraorbital, intracapsular, intraspinal or intrasternal, or topical administration to a bodily surface such as a mucosal membrane or epidermis.

The term “substantially free of sulfite compounds,” as used herein, is generally intended to refer to a formulation that contains markedly reduced amounts, minute amounts, or no amount, of sulfite compounds such that they neither function as a stabilizer nor reduce stability after terminal sterilization. Such sulfite compounds include, without limitation, sodium metabisulfite, sodium sulfite, sodium bisulfite or a mixture thereof.

The term “terminal sterilization” or “terminally sterilized,” as used herein, is generally intended to refer to sterilization of the final formulation, without using aseptic technique. For example, once the individual constituents of the formulation are combined in suitable concentrations to form a solution and the solution filled into suitable containers, the solution and the container may be subject to terminal sterilization. Terminal sterilization can be carried out by various methods that use, without limitation, heat including moist heat (or steam in an autoclave) and dry heat, irradiation, or gases such as ethylene oxide, etc.

The term “sparging,” as used herein, is generally intended to refer to the bubbling of a gas through a liquid to reduce or eliminate oxygen. The term “purging” as used herein is generally intended to refer to the process of using a flow of gas to sweep out another gas from a container or to use a flow of gas to form a blanket of an inert atmosphere over a container, process vessel, or space. This flow of a gas, such as nitrogen, substantially reduces the amount of oxygen in the purged space by excluding atmospheric air.

The term “substantially oxygen-free,” as used herein, is intended to generally refer to a solution or formulation that contains markedly reduced amounts or minute amounts, or no amount, of oxygen therein, preferably less than about 0.5 ppm. A substantially oxygen-free formulation generally will depend upon the rate and length of time by which the formulation is sparged with one or more inert gases that do not contain oxygen.

The term “substantially colorless,” as used herein, is intended to generally refer to a solution or formulation that has either a very little color, such as slight yellow, or a color which is not objectionable. Objectionable colors include dark yellow, pink, salmon, or another color that may proceed over time to brown.

The term “treating,” as used herein, is intended to generally refer to treating, relieving, reducing the severity of, or reducing the occurrence of disorders which may range in severity.

In the formulation of the present invention, levomepromazine or a pharmaceutically acceptable salt thereof is used in a concentration ranging from about 1 mg/ml to about 40 mg/ml of the formulation, preferably about 10 mg/ml to 30 mg/ml, more preferably 20 mg/ml. Of course, by varying the amounts of levomepromazine in the formulation or the amount of formulation administered, the composition may be administered at different dosages and to children and adults alike.

A pharmaceutically acceptable salt of levomepromazine includes, without limitation, maleate and hydrochloride salt forms. A pharmaceutically acceptable salt of EDTA, as used in a first stabilizer of the formulation, includes, without limitation, EDTA dipotassium, EDTA disodium, Mg, Cu, Zn, Fe, Ca salt of EDTA, EDTA tetrasodium and EDTA trisodium. A pharmaceutically acceptable salt of ascorbic acid, as used in a third stabilizer of the formulation, includes, without limitation, sodium ascorbate. A stabilizer that can be used as a second stabilizer in the formulation of the present invention includes, without limitation, monothioglycerol (MTG), cysteine, glutathione, and ethylgallate. An amount of each stabilizer in the formulation should be within the range of from about 0.001% to about 5%, preferably in the range of from about 0.05% to about 2% by weight per volume of the formulation. The combined amounts of stabilizers used in the present invention should be effective to stabilize the levomepromazine formulation of the present invention.

The selected combinations of these stabilizers, with sparging of the formulation, if necessary or desired, will reduce the decomposition of levomepromazine and the concentration of the decomposition products of levomepromazine in the formulation so that the formulation contains a concentration of a total impurities of less than about 3% by weight per volume of the formulation, or contains a concentration of the impurity levomepromazine sulfoxide of less than about 2% by weight per volume of the formulation. The formulation preferably is terminally sterilized. The formulations of the present invention maintain such levels of impurities during storage for at least three (3) months, desirably for at least six (6) months, more preferably for at least two (2) years, and most preferably for three (3) to five (5) years, at ambient conditions (under room temperature at normal humidity levels), or upon storage for at least two (2) months at 40° C. and 75% relative humidity.

EDTA or a pharmaceutically acceptable salt thereof generally is used in an amount ranging from about 0.001% to about 5%, preferably about 0.02% to about 0.2%, and more preferably 0.065% by weight per volume of the formulation. EDTA typically has been approved by the FDA for use in an amount (% w/v) ranging from about 0.025% to about 0.2% for intramuscular administration (IM) and in a concentration of about 0.2% for intravenous administration (IV).

When monothioglycerol (MTG) is selected as a stabilizer, it generally is used in an amount ranging from about 0.001% to about 5%, preferably about 0.05% to about 2%, and more preferably about 1% by weight per volume of the formulation. Monothioglycerol typically has been approved by the FDA for use in an amount (% w/v) ranging from about 0.5% to about 1% for intramuscular administration (IM) and ranging from about 0.5% to about 1% for intravenous administration (IV).

Where cysteine is selected as a stabilizer, it generally is used in an amount ranging from about 0.001% to about 5%, preferably about 0.07% to about 2.0%, and more preferably either 0.025% or 1% by weight per volume of the formulation. As the form of L-cysteine hydrochloride monohydrate, it can be used in a concentration ranging from about 0.02% to about 1% by weight per volume of the formulation. Cysteine hydrochloride typically has been approved by the FDA for use in an amount (% w/v) of about 0.1% for intramuscular administration (IM) and ranging from about 0.1% to about 2.6% for intravenous administration (IV).

Where glutathione is selected as a stabilizer, it generally is used in an amount ranging from about 0.001% to about 5%, and preferably about 0.02% to about 2% by weight per volume of the formulation. It is useful at an amount about 0.5% by weight per volume of the formulation in an intramuscular sustained release injection. Glutathione typically has been approved by the FDA for use in an amount (% w/v) of about 0.5% for intramuscular administration.

Where ethylgallate is selected as a stabilizer, it generally is used in an amount ranging from about 0.001% to about 5%, preferably about 0.02% to about 0.2%, and more preferably about 0.1% by weight per volume of the formulation. Ethylgallate is not very soluble in water and, therefore, an organic solvent such as alcohol, or a surfactant may be needed to dissolve ethylgallate in the formulation.

Ascorbic acid or a pharmaceutically acceptable salt thereof is generally used in an amount ranging from about 0.001% to about 5%, preferably about 0.05% to about 2%, and more preferably 0.1%, 0.25% or 1% by weight per volume of the formulation. Ascorbic acid typically has been approved by the FDA for use in an amount (% w/v) ranging from about 0.2% to about 1% for intramuscular administration (IM) and ranging from about 0.2% to about 2% for intravenous administration (IV).

The formulation of the present invention may be buffered to a desired pH to further promote stability and shelf life and to decrease formation of degradation products and/or objectionable discoloration of the formulation. To this end, a pH buffering agent may be included with the stabilizers. Conventional buffer systems or combinations known to regulate pH of a solution within a specific pH range, or at a particular pH, may be utilized in the formulation. The pH of the levomepromazine formulation of the present invention is buffered to a range from about 3 to about 7, preferably about 4 to about 5.5, and more preferably about 4.5. The formulation can include a citric acid buffer system, including pharmaceutically accepted salts of citric acid. One skilled in the art can adjust the amounts of citric acid and/or its acceptable salts to obtain a desired pH. For example, a combination of citric acid monohydrate in about 0.6% by weight per volume and sodium citrate dihydrate in about 1.2% weight by volume generally provides a pH of about 4 for the formulation. The same buffer system comprising citric acid monohydrate in an amount of about 0.06% by weight per volume and sodium citrate dihydrate in an amount of about 1% by weight per volume provides a formulation pH of about 4.5. Citric acid typically has been approved by the FDA for use in a concentration (% w/v) ranging from about 0.075% to about 2% for both intramuscular administration (IM) and intravenous administration (IV). Sodium citrate typically has been approved by the FDA for use in a concentration (% w/v) ranging from about 0.3% to about 6.6% for both IM and IV administration. Buffering formulations to a specific pH or narrow pH range helps to prevent precipitation of less soluble species such as the free base or salts and the degradation and formation of hydrolysis products or oxidative degradation of levomepromazine.

A formulation for parenteral administration is generally in the form of a solution, however, such formulations also generally can include emulsions, suspensions, creams, pastes, and other formulations that can be applied topically or by other non-oral administration methods. A medium for preparing solutions includes, without limitation, pure water, sterile isotonic saline or physiologically compatible organic solvents such as ethanol, 1,3-butanediol, 1, 2-propylene glycol, polyglycol(s) mixed with water, dimethyl sufoxide, fatty alcohols, triglycerides, Ringers solution, glucose and the like. The formulations of the present invention can be prepared using methods which are standard in the art, such as disclosed in REMINGTON'S PHARMACEUTICAL SCIENCES, 16^{th} Ed., Easton, Pa. (1980), disclosure of which is incorporated herein by reference in its entirety. The levomepromazine formulation of the present invention can be prepared by known methods. In an exemplary method of formulation, the individual components of the composition can be weighed out in accordance with the desired amount in the final formulation, and combined and mixed with a suitable medium, or individually mixed with the medium to form a parenterally administrable solution. The formulation further can comprise sodium chloride as a tonicity agent.

To further prevent oxidative degradation of levomepromazine in the final formulation, the formulation, once formed, can be sparged with an oxygen-free inert gas. Sparging removes or reduces the oxygen concentration in the solution. Inert gases that are biologically compatible and are free of oxygen, including, without limitation, nitrogen, carbon dioxide, argon, helium, and the like, and combinations thereof, can be used to sparge the solution. The rate of introduction of the gas into the solution, as well as the duration of time the solution is sparged, can vary as desired. Generally, however, rates of about 10 cc/min. to about 150 cc/min. over a period from about 10 seconds to about 90 minutes is generally sufficient, depending upon the volume of the formulation.

Oxygen concentration also can be reduced or eliminated by purging the formulation with a blanket of an inert oxygen-free gas, such as nitrogen. Purging generally removes oxygen from the headspace of the formulation, thereby eliminating oxygen from an environment otherwise accessible to the formulation itself and components therein. A combination of sparging and purging the formulation with an inert gas generally removes most of the oxygen from the formulation as well as from the headspace above the formulation. Purging can be done prior to and/or after filling the container. Sparging and purging, together, are particularly useful where the formulation has been contained in a container.

The final formulation also can be treated with terminal sterilization to provide a high assurance of sterility, thereby enhancing acceptability and confidence in the safety of the formulation. Terminal sterilization can use various methods including, without limitation, subjecting the formulation to heat, such as steam or dry heat, irradiation, or gasses such as ethylene oxide. Steam sterilization using moist heat under pressure, such as autoclaving, is the most dependable and preferable method for terminal sterilization in the present invention, Autoclaving can be done at least 120° C. for at least 15 minutes, preferably from 120° C. to 130° C., more preferably 121° C. to 123° C. for 15 to 20 minutes. Autoclaving can also been done at 134° C. for 3 minutes with a prevacuum high-pressure cycle. It has been discovered that the formulations of the present invention comprising selected stabilizers are not adversely affected when exposed to heat during terminal sterilization. Thus, the formulations can provide an assurance of high sterility in a range of from about 10⁶ to 10¹² (the probability of a microorganism surviving the autoclaving procedure is 10⁻⁶ to 10⁻¹²) after terminal sterilization such as autoclaving or other sterilization procedures involving heat. In a preferred embodiment, the formulations of the present invention are substantially free of a sulfite compound such as sodium sulfite, sodium metabisulfite or sodium bisulfite, and can withstand exposure to elevated temperatures without accelerating the formation of the degradation products of levomepromazine. The formulation comprising EDTA, MTG and ascorbic acid as a stabilizer is particularly preferred because of its stability after terminal sterilization such as autoclaving or other procedures involving heat. In addition, stability of such formulations is not solely or highly dependent on elimination of oxygen, and thus may eliminate the need for extensive sparging and/or purging, which is essential for previous levomepromazine formulations.

The formulation for parenteral administration of the present invention can be packed and/or stored in a suitable container, including, without limitation, syringes, ampoules, vials including sealed vials such as vials the openings of which are sealed with syringe pierceable septa or sure-seals caps, and the like. In one embodiment, the formulation is pre-filled in disposable syringes for self-administration by patients, with or without an auto-injector. Each container can contain a single dose of levomepromazine in a desired amount. For example, the container may contain 1 ml of a 25 mg/ml formulation, or it may contain 1 ml of a 20 mg/ml concentration of levomepromazine. To further minimize oxidative degradation of levomepromazine, the container could be filled with an inert gas, such as nitrogen and/or carbon dioxide, which is otherwise oxygen-free. It can be further contemplated that the container(s) be enclosed within a sealed package, from which oxygen has been included. This may be accomplished by vacuum packaging or by displacing oxygen with a blanket or purge of nitrogen, carbon dioxide, or other oxygen-free inert gas. After sealing the seal, the packaging materials themselves should be relatively impermeable to the diffusion of oxygen. Also, the packages should be opaque to ordinary light, as light induces the decomposition of levomepromazine. Standard methods for sealing and packaging the various containers described herein are well known in the art and can be used in conjunction with packaging and/or storing the compositions of the present invention.

Where the containers contain a single dosage amount, such as a 2 ml septum-sealed vial containing 1 ml of a formulation containing levomepromazine in a concentration of about 20 mg/ml, the formulation can be withdrawn simply by utilizing a syringe fitted with a needle, other injectable needle systems, or needleless injectable systems. During removal, however, care should be taken to administer the dose to the patient in a reasonably prompt manner to minimize the time the formulation is exposed to ambient oxygen and light.

The formulation of the present invention comprising levomepromazine or a pharmaceutically acceptable salt thereof can be used to treat a patient suffering from a disorder, which includes, without limitation, psychosis, agitation, pain, migraine headache, nausea, vomiting, itching, hypertension, benign prostatic hypertrophy, excess gastrointestinal (GI) secretions, or sleeplessness. In particular, pain can be moderate to severe pain which can result from the direct effects of diseases, such as cancer, diabetes, varicella zoster virus (shingles) infection, trauma, autoimmune disorders, and connective tissue diseases. Alternatively, pain can result as a consequence of disease treatment, as is often observed in patients with AIDS and cancer who receive chemotherapy, radiation therapy or surgery.

The formulation of the present invention is parenterally administered, e.g., the formulation is removed from its container, such as by being withdrawn into a syringe fitted with a needle, or other comparable administration systems, and administered by injection into the patient. For a primary initial treatment of psychosis, as a pre-medication for certain severe pain, and for the treatment of postoperative pain, levomepromazine is administered in doses ranging from about 40 mg/ml to about 100 mg/ml, given as three or four deep intramuscular injections. It is effective as a pre-medication or postoperative analgesic when parenterally administered in doses ranging from about 10 mg/ml to about 25 mg/ml every 8 hours, which is equivalent to about 20 mg to about 40 mg given orally. Typically, the last dose during pre-medication, given one hour before surgery, is usually about 25 mg/ml to about 50 mg/ml dosed via intramuscular injection.

Intravenous levomepromazine is primarily used as an infusion during surgery or labor. Effective doses typically range from about 10 mg to about 25 mg in 500 ml of a 5% glucose solution administered at a rate of about 20 to about 40 drops per minute.

Suitable dosage ranges of levomepromazine for use in treating other disorders can readily be determined by routine adjustment.

Similarly, parenteral doses of levomepromazine also can be administered to children. For example, an intramuscular dose of about 1/16 to about ⅛ mg/kg/day in one single dose, or administered among several injections, of a formulation having levomepromazine in a concentration of about 20 mg/ml, is generally effective. Typically, during surgery, for anesthetic purposes, 1/16 mg/kg dose in 250 ml of a 5% glucose solution can be administered as a slow infusion (20-50 drops/minute). The amount of levomepromazine administered generally will vary depending upon the patient characteristics including patient age, gender, size, weight, and medical history, as appreciated by those of ordinary skill in the art.

The amount of levomepromazine administered will generally vary depending upon the patient characteristics including patient age, gender, size, weight, and medical history, as appreciated by those of ordinary skill in the art. The amount of levomepromazine administered with the compositions of the present invention should be administered in accordance with generally accepted medical and pharmacy and government-approved dosing practices.

The invention will be further appreciated in light of the following examples.

### EXAMPLE 1

A series of exemplary stabilized levomepromazine formulations (Table 1) were prepared and tested for stability at To (initial time point) and at the 1-month time point as follows. Each formulation was buffered to a pH of about 4.5, utilizing a citric acid buffer. A buffer stock solution was prepared by weighing out sodium citrate dihydrate and citric acid monohydrate in appropriate proportions and dissolved in water for injection to a specified volume in a calibrated metric flask (herein designated as Solution A). The water may be sparged with oxygen-free gas prior to dissolution of the buffers. The excipients (stabilizers) to be included in the formulation were then individually weighed out (amount indicated in Table 1 as a % weight/volume) and dissolved in approximately 70% of the stock buffer solution to form Solution B. Levomepromazine HCl (about 2.2% w/v) was weighed out and added to Solution B after which Solution B was mixed until the levomepromazine dissolved. Solution B was then further diluted with stock buffer solution A to obtain the desired concentration of the ingredients (stabilizers and active drug compound) to form a final formulation, designated as Solution C. A portion of Solution C was dispensed into vials (non-sparged test samples designated with “X”). Another portion of Solution C was sparged (test samples designated with “v”) to remove or reduce oxygen and filled into purged vials. Each vial was sealed and capped prior to storage and/or further processing. Certain samples of each exemplary formulation was autoclaved with heat to terminally sterilize the formulation (test samples designated with “v”).

Sealed vials containing the exemplary formulations were then tested for stability as a function of time. The test involved removing samples from each vial at specific time intervals, such as at time 0 (“initial”) and 1 month, at ambient conditions (room temperature at normal humidity levels), to determine the levels of oxidative degradation products including sulfoxide, sulfone and other degradation products such as N-desmethyl levomepromazine in the solution. The degradation products (referred to in Table 1 as “impurities”) were individually and cumulatively quantified, and the concentrations of active levomepromazine in the sparged or non-sparged and autoclaved or non-autoclaved formulations were determined at the specified time period, as indicated in Table 1.

TABLE 1

| | | | | Degradation Products (% area) | | | | | | | |
|---|---|---|---|---|---|---|---|---|---|---|---|
| | | | Appearance | | | N- | | Largest | Total | | mOsmol/ |
| Excipients | Sparged | Autoclaved | Visual | Sulphoxide | Sulphone | Desmethyl | Levo | Unknown | Imp. | pH | kg |

RESULTS AT THE INITIAL TIME POINT (T₀)

| | | | | | | | | | | | |
|---|---|---|---|---|---|---|---|---|---|---|---|
| 0.065% EDTA | v | v | Pale Yellow | 0.15 | 0.01 | 0.02 | 99.77 | 0.03 | 0.23 | — | — |
| 1% Ascorbic Acid | x | v | Yellow | 0.51 | 0.06 | 0.03 | 99.16 | 0.08 | 0.84 | — | — |
| 0.5% Gluthathione | v | x | Very Pale | 0.09 | 0.01 | 0.04 | 99.78 | 0.03 | 0.22 | 3.84 | 351.00 |
| | | | Yellow | | | | | | | | |
| | x | x | Pale Yellow | 0.31 | 0.04 | 0.04 | 99.47 | 0.04 | 0.53 | 3.87 | 350.00 |
| 0.065% EDTA | v | v | Pale Yellow | 0.31 | — | 0.02 | 99.60 | 0.02 | 0.40 | — | — |
| 1% Mono-thioglycerol | x | v | Pale Yellow | 0.69 | — | 0.02 | 99.24 | 0.03 | 0.76 | — | — |
| 0.1% Ethyl Gallate | v | x | Very Pale | 0.05 | 0.02 | 0.05 | 99.84 | 0.03 | 0.16 | 4.51 | 376.00 |
| | | | Yellow | | | | | | | | |
| | x | x | Pale Yellow | 0.13 | 0.01 | 0.04 | 99.76 | 0.04 | 0.24 | 4.54 | 378.00 |
| 0.065% EDTA | v | v | Very Pale | 0.13 | 0.02 | 0.03 | 99.73 | 0.03 | 0.27 | — | — |
| 1% Ascorbic Acid | | | Yellow | | | | | | | | |
| | x | v | Very Pale | 0.44 | 0.06 | 0.03 | 99.33 | 0.05 | 0.67 | — | — |
| | | | Yellow | | | | | | | | |
| 1% Monothioglycerol | v | x | Very Pale | 0.07 | 0.02 | 0.05 | 99.81 | 0.03 | 0.19 | 3.99 | 431.00 |
| | | | Yellow | | | | | | | | |
| | x | x | Pale Yellow | 0.23 | 0.05 | 0.04 | 99.57 | 0.03 | 0.43 | 4.02 | 432.00 |
| 0.065% EDTA | v | v | Yellow | 0.23 | 0.03 | 0.03 | 99.56 | 0.05 | 0.44 | — | — |
| 1% Ascorbic Acid | x | v | Dark Yellow | 0.59 | 0.09 | 0.03 | 98.88 | 0.14 | 1.12 | — | — |
| 0.1% Ethyl Gallate | v | x | Pale Yellow | 0.09 | 0.02 | 0.04 | 99.77 | 0.03 | 0.23 | 3.98 | 336.00 |
| | x | x | Pale Yellow | 0.32 | 0.08 | 0.04 | 99.30 | 0.06 | 0.70 | 4.01 | 336.00 |
| 1% Ascorbic Acid | v | v | Yellow | 0.14 | 0.03 | 0.04 | 99.62 | 0.05 | 0.38 | — | — |
| 0.065% EDTA | x | v | Yellow | 0.54 | 0.10 | 0.04 | 98.85 | 0.17 | 1.15 | — | — |
| | v | x | Very Pale | 0.09 | 0.00 | 0.04 | 99.80 | 0.02 | 0.20 | 4.00 | 335.00 |
| | | | Yellow | | | | | | | | |
| | x | x | Pale Yellow | 0.46 | 0.07 | 0.04 | 99.06 | 0.09 | 0.94 | 4.03 | 337.00 |
| 0.25% Ascorbic Acid | v | v | Yellow | 0.09 | 0.04 | 0.04 | 99.60 | 0.05 | 0.40 | — | — |
| 0.065% EDTA | x | v | Yellow | 0.58 | 0.14 | 0.05 | 98.74 | 0.19 | 1.26 | — | — |
| | v | x | Very Pale | 0.08 | 0.03 | 0.04 | 99.69 | 0.05 | 0.31 | 4.35 | 295.00 |
| | | | Yellow | | | | | | | | |
| | x | x | Pale Yellow | 0.30 | 0.09 | 0.04 | 99.06 | 0.18 | 0.94 | 4.38 | 296.00 |
| 0.1% Ascorbic Acid | v | v | Yellow | 0.13 | 0.05 | 0.04 | 99.56 | 0.06 | 0.44 | — | — |
| 0.065% EDTA | x | v | Dark Yellow | 0.88 | 0.09 | 0.04 | 98.61 | 0.13 | 1.39 | — | — |
| | v | x | Very Pale | 0.09 | 0.03 | 0.05 | 99.74 | 0.03 | 0.26 | 4.46 | 286.00 |
| | | | Yellow | | | | | | | | |
| | x | x | Pale Yellow | 0.23 | 0.07 | 0.04 | 99.55 | 0.04 | 0.50 | 4.47 | 286.00 |

RESULTS AT THE ONE MONTH TIME POINT (T₀)

| | | | | | | | | | | | |
|---|---|---|---|---|---|---|---|---|---|---|---|
| 0.065% EDTA | v | v | Very Pale | 0.22 | 0.02 | 0.05 | 99.62 | 0.03 | 0.38 | — | — |
| 0.1% Cysteine | | | Yellow | | | | | | | | |
| | x | v | Pale | 0.78 | 0.02 | 0.06 | 98.93 | 0.05 | 1.07 | — | — |
| | | | Yellow/ | | | | | | | | |
| | | | Brown | | | | | | | | |
| | v | x | Colourless | 0.04 | 0.02 | 0.04 | 99.85 | 0.03 | 0.15 | 4.40 | 289.00 |
| | x | x | Colourless | 0.05 | 0.02 | 0.04 | 99.84 | 0.03 | 0.16 | 4.00 | 292.00 |
| 0.065% EDTA | v | v | Very Pale | 0.30 | 0.01 | 0.05 | 99.55 | 0.03 | 0.45 | — | — |
| 0.5% Gluthathione | | | Yellow | | | | | | | | |
| | x | v | Pale Yellow | 1.04 | — | 0.06 | 98.73 | 0.04 | 1.27 | — | — |
| | v | x | Colourless | 0.04 | 0.01 | 0.05 | 99.87 | 0.03 | 0.13 | 4.25 | 297.00 |
| | x | x | Colourless | 0.06 | 0.02 | 0.05 | 99.81 | 0.03 | 0.19 | 4.29 | 302.00 |
| 0.065% EDTA | v | v | Pale Yellow | 0.63 | — | 0.02 | 99.17 | 0.06 | 0.83 | — | — |
| 0.1% Ethyl Gallate | x | v | Yellow | 1.31 | — | 0.03 | 98.54 | 0.03 | 1.46 | — | — |
| | v | x | Very Pale | 0.04 | 0.01 | 0.04 | 99.86 | 0.03 | 0.14 | 4.51 | 280.00 |
| | | | Yellow | | | | | | | | |
| | x | x | Very pale | 0.04 | — | 0.04 | 99.86 | 0.04 | 0.14 | 4.55 | 281.00 |
| | | | Yellow | | | | | | | | |
| 0.065% EDTA | v | v | Colourless | 0.23 | 0.01 | 0.06 | 99.64 | 0.03 | 0.36 | — | — |
| 1% Mono-thioglycerol | x | v | Very Pale | 0.72 | 0.01 | 0.05 | 99.16 | 0.03 | 0.84 | — | — |
| | | | Yellow | | | | | | | | |
| | v | x | Colourless | 0.06 | 0.02 | 0.04 | 99.80 | 0.03 | 0.20 | 4.51 | 376.00 |
| | x | x | Very Pale | 0.18 | 0.02 | 0.04 | 99.70 | 0.03 | 0.30 | 4.55 | 376.00 |
| | | | Yellow | | | | | | | | |
| 0.065% EDTA | v | v | Very Pale | 0.23 | 0.01 | 0.06 | 99.60 | 0.03 | 0.40 | — | — |
| 0.025% Cysteine | | | Yellow | | | | | | | | |
| | x | v | Pale Yellow | 0.89 | 0.03 | 0.05 | 98.95 | 0.03 | 1.05 | — | — |
| | v | x | Colourless | 0.04 | 0.02 | 0.04 | 99.86 | 0.03 | 0.14 | 4.48 | 283.00 |
| | x | x | Colourless | 0.05 | 0.01 | 0.05 | 99.84 | 0.03 | 0.16 | 4.53 | 282.00 |

RESULTS AT THE INITIAL TIME POINT (T₀)

| | | | | | | | | | | | |
|---|---|---|---|---|---|---|---|---|---|---|---|
| 0.065% EDTA | v | v | Yellow | 0.23 | 0.03 | 0.03 | 99.56 | 0.05 | 0.44 | — | — |
| 1% Ascorbic Acid | X | v | Dark Yellow | 0.56 | 0.14 | 0.03 | 98.78 | 0.16 | 1.22 | — | — |
| 0.025% Cysteine | v | x | Pale Yellow | 0.10 | 0.03 | 0.04 | 99.73 | 0.03 | 0.27 | 3.95 | 336.00 |
| | X | x | Pale Yellow | 0.35 | 0.10 | 0.04 | 99.23 | 0.06 | 0.77 | 4.01 | 338.00 |
| 0.065% EDTA | v | v | Yellow | 0.20 | 0.02 | 0.03 | 99.63 | 0.04 | 0.37 | — | — |
| 1% Ascorbia Acid | X | v | Dark Yellow | 0.48 | 0.12 | 0.03 | 98.94 | 0.13 | 1.06 | — | — |
| 0.1% Cysteine | v | x | Very Pale | 0.11 | 0.02 | 0.05 | 99.72 | 0.02 | 0.28 | 3.89 | 344.00 |
| | | | Yellow | | | | | | | | |
| | X | x | Pale Yellow | 0.34 | 0.47 | 0.04 | 98.60 | 0.16 | 1.40 | 3.95 | 346.00 |
| 0.065% EDTA | v | v | Pale Yellow | 0.19 | 0.02 | 0.02 | 99.69 | 0.03 | 0.31 | — | — |
| 0.25% Ascorbic Acid | X | v | Yellow | 0.53 | 0.11 | 0.02 | 98.92 | 0.15 | 1.08 | — | — |
| 0.1% Cysteine | v | x | Pale Yellow | 0.09 | 0.03 | 0.04 | 99.74 | 0.03 | 0.26 | 4.30 | 297.00 |
| | X | x | Pale Yellow | 0.27 | 0.11 | 0.05 | 99.29 | 0.06 | 0.71 | 4.36 | 297.00 |
| 0.065% EDTA | v | v | Pale Yellow | 0.22 | 0.01 | 0.02 | 99.68 | 0.03 | 0.32 | — | — |
| 0.1% Ascorbic Acid | X | v | Dark Yellow | 0.85 | 0.01 | 0.04 | 98.87 | 0.07 | 1.13 | — | — |
| 0.1% Cysteine | v | x | Pale Yellow | 0.07 | 0.03 | 0.04 | 99.81 | 0.03 | 0.19 | 4.41 | 288.00 |
| | X | x | Pale Yellow | 0.22 | 0.08 | 0.04 | 99.48 | 0.04 | 0.52 | 4.44 | 290.00 |
| Drug Substance | v | v | Colourless | 0.26 | — | 0.07 | 99.63 | 0.04 | 0.37 | — | — |
| Alone | X | v | Colourless | 0.62 | — | 0.14 | 99.17 | 0.04 | 0.83 | — | — |
| | v | x | Colourless | 0.05 | — | 0.05 | 99.90 | — | 0.05 | 4.57 | 278.00 |
| | X | x | Colourless | 0.05 | — | 0.04 | 99.88 | 0.03 | 0.12 | 4.50 | 278.00 |
| Nozinan* | v | v | Colourless | 3.07 | 0.01 | 0.04 | 96.81 | 0.02 | 3.19 | ND | ND |
| | v | x | Colourless | 0.44 | 0.01 | 0.04 | 99.44 | 0.03 | 0.56 | — | — |

*Nozinan is thought to be sparged and the headspace filled by nitrogen to increase formulation stability.

RESULTS AT THE ONE MONTH TIME POINT (T_{1M})

| | | | | | | | | | | | |
|---|---|---|---|---|---|---|---|---|---|---|---|
| 0.065% EDTA | v | x | Pale Yellow | 0.10 | 0.03 | 0.04 | 99.49 | 0.07 | 0.51 | — | — |
| 1% Ascorbic Acid | x | x | Yellow | 0.52 | 0.11 | 0.06 | 98.51 | 0.13 | 1.49 | — | — |
| 0.5% Glutathione | | | | | | | | | | | |
| 0.065% EDTA | v | x | Pale Yellow | 0.48 | 0.02 | 0.04 | 99.18 | 0.06 | 0.82 | — | — |
| 1% Mono-thioglycerol | x | x | Yellow | 2.33 | 0.02 | 0.06 | 97.31 | 0.08 | 2.69 | — | — |
| 0.1% Ethyl Gallate | | | | | | | | | | | |
| 0.065% EDTA | v | x | Colourless | 0.09 | 0.03 | 0.04 | 99.50 | 0.07 | 0.50 | — | — |
| 1% Ascorbic Acid | x | x | Very Pale | 0.32 | 0.05 | 0.04 | 99.15 | 0.09 | 0.85 | — | — |
| 1% Monothioglycerol | | | Yellow | | | | | | | | |
| 0.065% EDTA | v | x | Yellow | 0.15 | 0.03 | 0.04 | 99.43 | 0.07 | 0.57 | — | — |
| 1% Ascorbic Acid | x | x | Orange | 0.63 | 0.07 | 0.05 | 98.61 | 0.18 | 1.39 | — | — |
| 0.1% Ethyl Gallate | | | | | | | | | | | |
| 1% Ascorbic Acid | v | x | Yellow | 0.17 | 0.04 | 0.04 | 99.28 | 0.07 | 0.72 | — | — |
| 0.065% | x | x | Orange | 0.69 | 0.09 | 0.06 | 98.23 | 0.21 | 1.77 | — | — |
| 0.25% Ascorbic Acid | v | x | Dark Yellow | 0.08 | 0.04 | 0.04 | 99.39 | 0.07 | 0.61 | — | — |
| 0.065% EDTA | x | x | Brown | 0.77 | 0.18 | 0.06 | 97.65 | 0.44 | 2.35 | — | — |
| 0.1% Ascorbic Acid | v | x | Orange | 0.21 | 0.07 | 0.04 | 98.98 | 0.17 | 1.02 | — | — |
| 0.065% EDTA | X | x | Brown | 1.49 | 0.18 | 0.05 | 97.00 | 0.47 | 3.00 | — | — |
| 0.065% EDTA | v | x | Very Pale | 0.54 | 0.02 | 0.04 | 99.12 | 0.06 | 0.88 | — | — |
| 0.1% Cysteine | | | Yellow | | | | | | | | |
| | X | x | Yellow | 3.01 | 0.04 | 0.05 | 96.44 | 0.06 | 3.56 | — | — |
| 0.065% EDTA | v | x | Very Pale | 0.72 | 0.02 | 0.04 | 98.95 | 0.07 | 1.05 | — | — |
| 0.5% Glutathione | | | Yellow/ | | | | | | | | |
| | | | colourless | | | | | | | | |
| | X | x | Yellow | 2.61 | 0.04 | 0.06 | 96.85 | 0.08 | 3.15 | — | — |
| 0.065% EDTA | v | x | Yellow | 0.70 | 0.02 | 0.04 | 98.44 | 0.07 | 1.56 | — | — |
| 0.1% Ethyl Gallate | X | x | Yellow | 1.99 | 0.03 | 0.05 | 97.59 | 0.07 | 2.41 | — | — |
| 0.065% EDTA | v | x | Colourless | 0.58 | 0.02 | 0.04 | 99.06 | 0.06 | 0.94 | — | — |
| 1% Mono-thioglycerol | X | x | Very Pale | 2.37 | 0.05 | 0.07 | 97.05 | 0.06 | 2.95 | — | — |
| | | | Yellow/ | | | | | | | | |
| | | | Colourless | | | | | | | | |
| 0.065% EDTA | v | x | Very Pale | 0.86 | 0.00 | 0.04 | 98.78 | 0.07 | 1.22 | — | — |
| 0.025% Cysteine | | | Yellow | | | | | | | | |
| | X | x | Pale Yellow | 2.07 | 0.00 | 0.04 | 97.64 | 0.08 | 2.36 | — | — |
| 0.065% EDTA | v | x | Yellow | 0.14 | 0.03 | 0.04 | 99.40 | 0.07 | 0.60 | — | — |
| 1% Ascorbic Acid | x | x | Orange | 0.59 | 0.08 | 0.05 | 98.58 | 0.17 | 1.42 | — | — |
| 0.025% Cysteine | | | | | | | | | | | |
| 0.065% EDTA | v | x | Yellow | 0.14 | 0.03 | 0.04 | 99.40 | 0.07 | 0.60 | — | — |
| 1% Ascorbic Acid | x | x | Dark Yellow | 0.58 | 0.08 | 0.05 | 98.66 | 0.14 | 1.34 | — | — |
| 0.1% Cysteine | | | | | | | | | | | |
| 0.065% EDTA | v | x | Yellow | 0.18 | 0.04 | 0.04 | 99.27 | 0.07 | 0.73 | — | — |
| 0.25% Ascorbic Acid | x | x | Brown | 0.67 | 0.14 | 0.05 | 98.17 | 0.25 | 1.83 | — | — |
| 0.1% Cysteine | | | | | | | | | | | |
| 0.065% EDTA | v | x | Yellow | 0.18 | 0.04 | 0.04 | 99.25 | 0.07 | 0.75 | — | — |
| 0.1% Ascorbic Acid | x | x | Brown | 1.31 | 0.15 | 0.05 | 97.46 | 0.27 | 2.54 | — | — |
| 0.1% Cysteine | | | | | | | | | | | |
| Drug Substance | v | x | Pale Pink | 0.42 | 0.02 | 0.05 | 99.25 | 0.07 | 0.75 | — | — |
| Alone | x | x | Pale Pink* | 0.89 | 0.00 | 0.06 | 98.76 | 0.09 | 1.24 | — | — |
| Nozinam | v | x | Colourless | 0.15 | 0.00 | 0.03 | 99.54 | 0.14 | 0.46 | — | — |

*more colored than sparged

In Table 1, the formulations comprising selected stabilizers were compared to a control sample (designated as “drug substance alone”), i.e., a formulation of levomepromazine having no stabilizers. Table 1 shows the beneficial stability of the formulations of the present invention in comparison with the control sample with respect to decreased impurities and discoloration over the time period. Such an improvement in the stability is particularly useful for obtaining a regulatory approval for a formulation for parenteral administration of levomepromazine. In addition, the formulations of the present invention show the advantages of enhancing formulation stability while minimizing discoloration caused by autoclaving. Particularly, it is worth noting that Nozinan®, containing sodium sulfite, was found to be susceptible to autoclave treatment and could not withstand the heat. More specifically, the levomepromazine sulfoxide impurity in Nozinan® escalated upon exposure to high heat, causing the Nozinan® formulation to exceed the maximum impurity limit (generally about 3%) typically required for government approval.

Similarly, Table 2 tabulates the stability test results of selected exemplary formulations at specific time intervals, i.e., at time 0 (“initial”), at 1 month, and at 2 month time periods, at 40° Centigrade and 75% relative humidity. The degradation products were individually and cumulatively quantified, and the concentration of active levomepromazine in formulations either sparged or un-sparged (all formulations were not autoclaved) and at the specified time period was determined in the same manner as the samples of Table 1. As shown in Table 2, beneficial advantages in stability and long term stabilizing effects may be gained with the levomepromazine formulation comprising selected stabilizers as described in the present invention, particularly with respect to decreased decomposition and discoloration over time. Particularly, it is apparent that sparging and purging with an oxygen-free gas to remove oxygen from both the levomepromazine solution as well as the headspace enhances long-term stability.

TABLE 2
NON-AUTOCLAVED STABILITY RESULTS AT 40° C./75% RELATIVE HUMIDITY
ON SELECTED FORMULATIONS AT T₀, T_{1M} AND T_{2M} TIME POINTS

| | | | | | Degradation Products (% area) | | | | | | |
|---|---|---|---|---|---|---|---|---|---|---|---|
| | | | | | | | | | Largest | | |
| | | | | Appearance | | | N- | | Un- | Total | Total ICH |
| Excipients | Sparged | Autoclaved | Time | Visual | Sulphoxide | Sulphone | Desmethyl | Levo | known | Imp. | Impurities |
| 0.065% EDTA | v | x | Initial | v. pale | 0.09 | 0.01 | 0.04 | 99.78 | 0.03 | 0.22 | — |
| 1% ascorbic acid | | | | yellow | | | | | | | |
| | v | x | 1 month | Pale yellow | 0.10 | 0.03 | 0.04 | 99.49 | 0.07 | 0.51 | — |
| 0.5% Gluthathione | v | x | 2 month | Pale yellow | 0.18 | 0.04 | 0.04 | 99.30 | 0.07 | 0.70 | 0.18 |
| 0.065% EDTA | x | x | Initial | Pale yellow | 0.31 | 0.04 | 0.04 | 99.47 | 0.04 | 0.53 | — |
| 1% ascorbic acid | x | x | 1 month | Yellow | 0.52 | 0.11 | 0.06 | 98.51 | 0.13 | 1.49 | — |
| 0.5% Glutathione | x | x | 2 month | Yellow | 0.55 | 0.09 | 0.04 | 98.66 | 0.12 | 1.34 | 0.87 |
| 0.065% EDTA | v | x | Initial | v. pale | 0.07 | 0.02 | 0.05 | 99.81 | 0.03 | 0.19 | — |
| 1% ascorbic acid | | | | yellow | | | | | | | |
| 1% monothioglycerol | v | x | 1 month | Colourless | 0.09 | 0.03 | 0.04 | 99.50 | 0.07 | 0.50 | — |
| | v | x | 2 month | Colourless/ | 0.07 | 0.03 | 0.04 | 99.52 | 0.07 | 0.48 | 0.00 |
| | | | | very pale | | | | | | | |
| | | | | yellow | | | | | | | |
| 0.065% EDTA | x | x | Initial | Pale yellow | 0.23 | 0.05 | 0.04 | 99.57 | 0.03 | 0.43 | — |
| 1% ascorbic acid | x | x | 1 month | Very pale | 0.32 | 0.05 | 0.04 | 99.15 | 0.09 | 0.85 | — |
| 1% monothioglycerol | | | | yellow | | | | | | | |
| | x | x | 2 month | Pale yellow | 0.28 | 0.07 | 0.04 | 99.12 | 0.09 | 0.88 | 0.28 |
| Drug substance | v | x | Initial | Colourless | 0.05 | ND | 0.05 | 99.90 | ND | 0.05 | — |
| Alone | v | x | 1 month | Pale pink | 0.42 | 0.02 | 0.05 | 99.25 | 0.07 | 0.75 | — |
| | v | x | 2 month | Pale pink | 0.79 | 0.02 | 0.06 | 98.83 | 0.08 | 1.17 | 0.79 |
| Drug substance | x | x | Initial | Colourless | 0.05 | ND | 0.04 | 99.88 | 0.03 | 0.12 | — |
| Alone | x | x | 1 month | Pale pink* | 0.89 | 0.00 | 0.06 | 98.76 | 0.09 | 1.24 | — |
| | x | x | 2 month | Pale pink* | 1.75 | 0.03 | 0.08 | 97.77 | 0.10 | 2.23 | 1.85 |
| Nozinan | v | x | Initial | Colourless | 0.44 | 0.01 | 0.04 | 99.44 | 0.03 | 0.56 | — |
| | v | x | 1 month | Colourless | 0.15 | 0.00 | 0.03 | 99.54 | 0.14 | 0.46 | — |
| | v | x | 2 month | Colourless | 0.06 | 0.02 | 0.03 | 99.61 | 0.09 | 0.39 | 0.00 |
| 0.065% EDTA | v | x | Initial | Colourless | 0.04 | 0.02 | 0.04 | 99.85 | 0.03 | 0.15 | |
| 0.1% Cysteine | v | x | 1 month | Very Pale | 0.54 | 0.02 | 0.04 | 99.12 | 0.06 | 0.88 | |
| | | | | yellow | | | | | | | |
| | v | x | 2 month | Pale Yellow | 2.15 | 0.03 | 0.05 | 97.41 | 0.07 | 2.59 | 2.15 |
| 0.065% EDTA | x | x | Initial | Colourless | 0.05 | 0.02 | 0.04 | 99.84 | 0.03 | 0.16 | |
| 0.1% Cysteine | x | x | 1 month | Yellow | 3.01 | 0.04 | 0.05 | 96.44 | 0.06 | 3.56 | |
| | x | x | 2 month | Yellow | 4.76 | 0.02 | 0.05 | 94.83 | 0.06 | 5.17 | 4.76 |
| 0.065% EDTA | v | x | Initial | Colourless | 0.04 | 0.01 | 0.05 | 99.87 | 0.03 | 0.13 | |
| 0.5% Glutathione | v | x | 1 month | Very Pale | 0.72 | 0.02 | 0.04 | 98.95 | 0.07 | 1.05 | |
| | | | | Yellow/ | | | | | | | |
| | | | | colourless | | | | | | | |
| | v | x | 2 month | Very Pale | 1.06 | 0.03 | 0.05 | 98.46 | 0.07 | 1.54 | 1.06 |
| | | | | Yellow | | | | | | | |
| 0.065% EDTA | x | x | Initial | Colourless | 0.06 | 0.02 | 0.05 | 99.81 | 0.03 | 0.19 | |
| 0.5% Glutathione | x | x | 1 month | Yellow | 2.61 | 0.04 | 0.06 | 96.85 | 0.08 | 3.15 | |
| | x | x | 2 month | Yellow | 4.12 | 0.04 | 0.05 | 95.29 | 0.11 | 4.71 | 4.23 |
| 0.065% EDTA | v | x | Initial | Colourless | 0.06 | 0.02 | 0.04 | 99.80 | 0.03 | 0.20 | |
| 1% monoglycerol | v | x | 1month | Colourless | 0.58 | 0.02 | 0.04 | 99.06 | 0.06 | 0.94 | |
| | v | x | 2 month | Very pale | 0.67 | 0.02 | 0.04 | 98.94 | 0.07 | 1.06 | 0.67 |
| | | | | yellow/ | | | | | | | |
| | | | | colourless | | | | | | | |
| 0.065% EDTA | x | x | Initial | v. pale | 0.18 | 0.02 | 0.04 | 99.70 | 0.03 | 0.30 | |
| 1% monoglycerol | | | | yellow | | | | | | | |
| | x | x | 1 month | Very pale | 2.37 | 0.05 | 0.07 | 97.05 | 0.06 | 2.95 | |
| | | | | yellow/ | | | | | | | |
| | | | | colourless | | | | | | | |
| | x | x | 2 month | Very pale | 2.30 | 0.03 | 0.06 | 97.25 | 0.06 | 2.75 | 2.30 |
| | | | | yellow/ | | | | | | | |
| | | | | colourless | | | | | | | |

*More colored than sparged
ND Not detected
\hat{ }100 - Levo %

### EXAMPLE 2

An exemplary stabilized formulation of levomepromazine (ingredients and amounts shown in Table 3) was prepared and tested in an eight-week stability study (Table 4). The formulation prepared with components and amounts shown in Table 3 is as follows: (1) sodium citrate dihydrate and citric acid monohydrate were weighed out and transferred to a mixing vessel containing sparged water for injection (WFI) (volume of WFI required: approximately 70% of the final volume of solution). The buffer solution was mixed until the citric acid dissolved. Disodium edetate, ascorbic acid, and monothioglycerol, were all individually weighed out and added to and mixed with the buffer solution in the vessel. The solution was mixed until all the components dissolved. The pH was measured, and found to be about 4.7. Levomepromazine Hydrochloride was weighed out and transferred to the mixing vessel. The solution was mixed until the levomepromazine completely dissolved. The pH of the solution was measured, and found to be about 4.5. The solution then was sparged with an oxygen-free inert gas to reduce but not eliminate the amount of dissolved oxygen and filled into vials. To fill the vials, 1 ml of the solution was dispensed in a 2 ml vial under oxygen-free or reduced oxygen conditions, and the vials were stoppered and capped. The headspace above the solution was blanketed with nitrogen or argon to protect the formulation from exposure to oxygen prior to and during capping. The formulation in each vial contained levomepromazine in a concentration of about 20 mg/ml, had a pH of about 4.5, and an osmolality of about 322 milliosmoles/kg.

TABLE 3

Formula

| Materials | % w/v | Quantity (mg) |
|---|---|---|
| Levomepromazine HCl | 2.222 | 22.22 |
| Disodium Edetate (EDTA) | 0.065 | 0.65 |
| Ascorbic Acid | 1.000 | 10.00 |
| Monothioglycerol (MTG) | 1.000 | 10.00 |
| Citric Acid Monohydrate | 0.059 | 0.57 |
| Sodium Citrate Dihydrate | 1.094 | 10.94 |
| WFI | to 100% | to 1 ml |

The eight-week stability results of the formulation shown in Table 3 are provided in Table 4.

TABLE 4
STABILITY DATA FOR THE INITIAL AND 3 MONTH TIME POINT

| | | MTG | Ascorbic | EDTA | | | | | | |
|---|---|---|---|---|---|---|---|---|---|---|
| | | Content | Acid | Content | Levomepromazine | HPLC DEGRADATION PRODUCTS (% nominal) | | | | |
| | Storage | (% | Content | (% | Content | | | Largest | Total | Total |
| | Condition | nominal) | (% nominal) | nominal) | (% nominal) | Impurity 1 | Impurity 2 | unknown | unknowns | Impurities |
| | Specification | NMT | NMT | NMT | 95.0-105.0 | ≦2.0 | ≦0.2 | 0.2 | ≦.1.0 | ≦3.0 |
| | release | 110% | 110% | 110% | | (≦0.5) | | | (≦0.5) | (≦1.0) |
| | specification in | | | | | | | | | |
| | jackets if | | | | | | | | | |
| | application | | | | | | | | | |
| | Initial | 81 | 83 | 101 | 101.1 | 0.3 | ND | 0.2 | 0.2 | 0.5 |
| 3 Month | 40° C. (Inverted) | 83 | 85 | 95 | 99.1 | 0.1 | ND | ND | ND | 0.1 |
| | 40° C. (Inverted) | 75 | 69 | 90 | 98.9 | 0.1 | ND | ND | ND | 0.1 |
| | 40° C. (Upright) | 65 | 71 | 92 | 99.6 | 0.1 | ND | ND | ND | 0.1 |
| | 25° C. (Inverted) | 81 | 81 | 92 | 99.2 | 0.1 | ND | ND | ND | 0.1 |
| | 25° C. (upright) | NT | NT | NT | NT | NT | NT | NT | NT | NT |

MTG: Monothioglycerol
EDTA: Ethylenediaminetetra-Acetic Acid Disodium Salt
Impurity 1: Sulphoxide degradation product
Impurity 2: Sulphone degradation product
NMT: Not more than
ND: None detected above 0.1% by wt

As shown in Table 4, levomepromazine formulations having a combination of EDTA in a concentration of about 0.065% by weight per volume, ascorbic acid in a concentration of about 1% by weight per volume, and monothioglycerol in a concentration of about 1% by weight per volume, showed excellent stability, lack of objectionable discoloration, and minimal formation of degradation products.

While the present invention has been illustrated by the description of embodiments thereof, and while the embodiments have been described in considerable detail, it is not intended to restrict or in any way limit the scope of the appended claims to such detail. Additional advantages and modifications will be readily apparent to those skilled in the art. The invention, in its broader aspects, is not limited, therefore, to the specific details, representative apparatus and method, and illustrated examples described. Accordingly, departures may be made from such details without departing from the scope or spirit of Applicant's generally inventive concept.

1. A formulation comprising:
(a) a therapeutically effective amount of levomepromazine or a pharmaceutically acceptable salt thereof,
(b) ethylenediaminetetraacetic acid (EDTA) or a pharmaceutically acceptable salt thereof as a first stabilizer, and
(c) monothioglycerol (MTG) or glutathione as a second stabilizer,
wherein said stabilizers are present in an amount effective to stabilize said formulation, and wherein said formulation is subjected to sparging.

2. The formulation of claim 1, wherein said second stabilizer is MTG.

3. The formulation of claim 1, wherein said second stabilizer is glutathione.

4. The formulation of claim 1, wherein said formulation is substantially free of sulfite compounds.

5. The formulation of claim 1, wherein said formulation is terminally sterilized.

6. The formulation of claim 5, wherein said formulation is terminally sterilized by autoclaving.

7. The formulation of claim 1, wherein each of said stabilizers is present in an amount of from about 0.001% to about 5% by weight per volume of the formulation.

8. The formulation of claim 7, wherein said amount of EDTA or a pharmaceutically acceptable salt thereof is about 0.02% to about 0.2% by weight per volume of the formulation.

9. The formulation of claim 7, wherein said amount of MTG or glutathione is about 0.05% to about 2% by weight per volume of the formulation.

10. The formulation of claim 1, wherein said formulation contains a concentration of total impurities of less than about 3% by weight per volume of the formulation.

11. The formulation of claim 1, wherein said formulation contains a concentration of the impurity levomepromazine sulfoxide of less than about 2% by weight per volume of the formulation.

12. The formulation of claim 5, wherein said formulation contains a concentration of total impurities of less than about 3% by weight per volume of the formulation.

13. The formulation of claim 5, wherein said formulation contains a concentration of the impurity levomepromazine sulfoxide of less than about 2% by weight per volume of the formulation.

14. The formulation of claim 1, further comprising a pH buffering agent in an amount sufficient to buffer the formulation to a pH in the range of from about 3 to about 7.

15. The formulation of claim 14, wherein said pH is in the range of from about 4 to about 5.5.

16. The formulation of claim 14, wherein said pH buffering agent comprises citric acid or a pharmaceutically acceptable salt thereof.

17. The formulation of claim 1, wherein said formulation is substantially oxygen-free.

18. The formulation of claim 1, wherein said formulation is suitable for parenteral administration.

19. The formulation of claim 18, wherein said parenteral administration comprises injection.

20. The formulation of claim 18, wherein said formulation is contained in a container selected from the group consisting of a vial, a syringe and an ampoule.

21. A formulation comprising:
(a) a therapeutically effective amount of levomepromazine or a pharmaceutically acceptable salt thereof,
(b) ethylenediaminetetraacetic acid (EDTA) or a pharmaceutically acceptable salt thereof as a first stabilizer,
(c) monothioglycerol (MTG) or glutathione as a second stabilizer, and
(d) ascorbic acid or a pharmaceutically acceptable salt thereof as a third stabilizer,
wherein said stabilizers are present in an amount effective to stabilize said formulation.

22. The formulation of claim 21, wherein said formulation is subjected to sparging.

23. The formulation of claim 21, wherein said second stabilizer is MTG.

24. The formulation of claim 21, wherein said second stabilizer is glutathione.

25. The formulation of claim 21, wherein said formulation is substantially free of sulfite compounds.

26. The formulation of claim 21, wherein said formulation is terminally sterilized.

27. The formulation of claim 26, wherein said formulation is terminally sterilized by autoclaving.

28. The formulation of claim 21, wherein each of said stabilizers is present in an amount of from about 0.001% to about 5% by weight per volume of the formulation.

29. The formulation of claim 28, wherein said amount of EDTA or pharmaceutically acceptable salt thereof is about 0.02% to about 0.2% by weight per volume of the formulation.

30. The formulation of claim 28, wherein said amount of MTG, glutathione, or ascorbic acid or a pharmaceutically acceptable salt thereof is about 0.05% to about 2% by weight per volume of the formulation.

31. The formulation of claim 21, wherein said formulation contains a concentration of total impurities of less than about 3% by weight per volume of the formulation.

32. The formulation of claim 21, wherein said formulation contains a concentration of the impurity levomepromazine sulfoxide of less than about 2% by weight per volume of the formulation.

33. The formulation of claim 26, wherein said formulation contains a total concentration of impurities of less than about 3% by weight per volume of the formulation.

34. The formulation of claim 26, wherein said formulation contains a total concentration of the impurity levomepromazine sulfoxide of less than about 2% by weight per volume of the formulation.

35. The formulation of claim 21, further comprising a pH buffering agent in an amount sufficient to buffer the formulation to a pH in the range of from about 3 to about 7.

36. The formulation of claim 35, wherein said pH is in the range of from about 4 to about 5.5.

37. The formulation of claim 35, wherein said pH buffering agent comprises citric acid or a pharmaceutically acceptable salt thereof.

38. The formulation of claim 21, wherein said formulation is substantially oxygen-free.

39. The formulation of claim 21, wherein said formulation is suitable for parenteral administration.

40. The formulation of claim 39, wherein said parenteral administration comprises injection.

41. The formulation of claim 39, wherein said formulation is contained in a container selected from the group consisting of a syringe, a vial and an ampoule.

42. A formulation comprising:
(a) a therapeutically effective amount of levomepromazine or a pharmaceutically acceptable salt thereof,
(b) ethylenediaminetetraacetic acid (EDTA) or a pharmaceutically acceptable salt thereof as a first stabilizer,
(c) ethylgallate or cysteine as a second stabilizer, and
(d) ascorbic acid or a pharmaceutically acceptable salt thereof as a third stabilizer,
wherein said stabilizers are present in an amount effective to stabilize said formulation, and wherein said formulation is subjected to sparging.

43. The formulation of claim 42, wherein said second stabilizer is ethylgallate.

44. The formulation of claim 42, wherein said second stabilizer is cysteine.

45. The formulation of claim 42, wherein said formulation is substantially free of sulfite compounds.

46. The formulation of claim 42, wherein said formulation is terminally sterilized.

47. The formulation of claim 46, wherein said formulation is terminally sterilized by autoclaving.

48. The formulation of claim 42, wherein each of said stabilizers is present in an amount of from about 0.001% to about 5% by weight per volume of the formulation.

49. The formulation of claim 48, wherein said amount of EDTA or a pharmaceutically acceptable salt thereof is about 0.02% to about 0.2% by weight per volume of the formulation.

50. The formulation of claim 48, wherein said amount of ethylgallate, cysteine, or ascorbic acid or a pharmaceutically acceptable salt thereof is about 0.05% to about 2% by weight per volume of the formulation.

51. The formulation of claim 42, wherein said formulation contains a concentration of total impurities of less than about 3% by weight per volume of the formulation.

52. The formulation of claim 42, wherein said formulation contains a concentration of the impurity levomepromazine sulfoxide of less than about 2% by weight per volume of the formulation.

53. The formulation of claim 46, wherein said formulation contains a concentration of total impurities of less than about 3% by weight per volume of the formulation.

54. The formulation of claim 46, wherein said formulation contains a concentration of the impurity levomepromazine sulfoxide of less than about 2% by weight per volume of the formulation.

55. The formulation of claim 42, further comprising a pH buffering agent in an amount sufficient to buffer the formulation to a pH in the range of from about 3 to about 7.

56. The formulation of claim 55, wherein said pH is in the range of from about 4 to about 5.5.

57. The formulation of claim 55, wherein said pH buffering agent comprises citric acid or a pharmaceutically acceptable salt thereof.

58. The formulation of claim 42, wherein the formulation is substantially oxygen-free.

59. The formulation of claim 42, wherein said formulation is suitable for parenteral administration.

60. The formulation of claim 59, wherein said parenteral administration comprises injection.

61. The formulation of claim 59, wherein said formulation is contained in a container selected from the group consisting of a syringe, a vial and an ampoule.

62. The formulation of claim 21, wherein
said therapeutically effective amount of levomepromazine or a pharmaceutically acceptable salt thereof is about 10 mg/ml to about 30 mg/ml of the formulation,
EDTA or a pharmaceutically acceptable salt thereof is in an amount of about 0.02% to about 0.2% by weight per volume of the formulation,
MTG is in an amount of about 0.05% to 2.0% by weight per volume of the formulation, and
ascorbic acid or a pharmaceutically acceptable salt thereof is an amount of about 0.05% to about 2.0% by weight per volume of the formulation.

63. A formulation comprising
levomepromazine HCl in an amount of about 2.2% by weight per volume of the formulation;
ascorbic acid in an amount of about 1% by weight per volume of the formulation;
EDTA in an amount of about 0.065% by weight per volume of the formulation; and
MTG in an amount of about 1% by weight per volume of the formulation.

64. A stable terminally sterilized formulation comprising a therapeutically effective amount of levomepromazine or a pharmaceutically acceptable salt thereof, wherein said formulation contains a concentration of total impurities of less than about 3% by weight per volume of the formulation and is terminally sterillized.

65. The formulation of claim 64, wherein said concentration of the impurity levomepromazine sulfoxide is less than about 2% by weight per volume of the formulation.

66. A method for stabilizing a formulation of levomepromazine, comprising:
(a) combining a therapeutically effective amount of levomepromazine or a pharmaceutically acceptable salt thereof, and a stabilizing amount of a combination of stabilizers in a medium to form a formulation, and
(b) sparging said formulation with an oxygen-free inert gas,
wherein said combination of stabilizers comprises (1) EDTA or a pharmaceutically acceptable salt thereof as a first stabilizer and (2) monothioglycerol (MTG) or glutathione as a second stabilizer.

67. The method of claim 66, further comprising subjecting said formulation to terminal sterilization.

68. The method of claim 67, wherein said terminal sterilization comprises autoclaving.

69. The method of claim 66, wherein said gas is selected from the group consisting of nitrogen, carbon dioxide, argon, helium, and combinations thereof.

70. The method of claim 66, further comprising adding to said formulation obtained from a step (a), a pH buffering agent in an amount sufficient to buffer the formulation to a pH in the range of from about 3 to about 7.

71. The method of claim 70, wherein said pH is in the range of from about 4 to about 5.5.

72. The method of claim 70, said pH buffering agent comprises citric acid or a pharmaceutically acceptable salt thereof.

73. The method of claim 66, wherein said medium is selected from the group consisting of purified water, glucose, sterile isotonic saline, physiologically compatible organic solvents and a mixture thereof.

74. A method for stabilizing a formulation of levomepromazine, comprising:
combining a therapeutically effective amount of levomepromazine or a pharmaceutically acceptable salt thereof, and a stabilizing amount of a combination of stabilizers in a medium suitable for parenteral administration to form a formulation,
wherein said combination of stabilizers comprises (1) EDTA or a pharmaceutically acceptable salt thereof as a first stabilizer, (2) monothioglycerol (MTG) or glutathione as a second stabilizer, and (3) ascorbic acid or a pharmaceutically acceptable salt thereof as a third stabilizer.

75. The method of claim 74, further comprising subjecting said formulation to sparging with an oxygen-free inert gas.

76. The method of claim 74 or 75, further comprising subjecting said formulation to terminal sterilization.

77. The method of claim 76, wherein said terminal sterilization comprises autoclaving.

78. The method of claim 75, wherein said gas is selected from the group consisting of nitrogen, carbon dioxide, argon, helium, and combinations thereof.

79. The method of claim 74, further comprising adding to said formulation a pH buffering agent in an amount sufficient to buffer the formulation to a pH in the range of from about 3 to about 7.

80. The method of claim 79, wherein said pH is in the range of from about 4 to about 5.5.

81. The method of claim 79, wherein said pH buffering agent comprises citric acid or a pharmaceutically acceptable salt thereof.

82. The method of claim 74, wherein said medium is selected from the group consisting of purified water, glucose, sterile isotonic saline, physiologically compatible organic solvents and a mixture thereof.

83. A method for stabilizing a formulation of levomepromazine, comprising:
(a) combining a therapeutically effective amount of levomepromazine or a pharmaceutically acceptable salt thereof and a stabilizing amount of a combination of stabilizers in a medium suitable for parenteral administration to form a formulation, and
(b) subjecting said formulation to sparging with an oxygen-free inert gas, wherein said combination of stabilizers comprises (1) EDTA or a pharmaceutically acceptable salt thereof as a first stabilizer, (2) ethylgallate or cysteine as a second stabilizer, and (3) ascorbic acid or a pharmaceutically acceptable salt thereof as a third stabilizer.

84. The method of claim 83, further comprising subjecting said formulation to terminal sterilization.

85. The method of claim 84, wherein said terminal sterilization comprises autoclaving.

86. The method of claim 83, wherein said gas is selected from the group consisting of nitrogen, carbon dioxide, argon, helium, and combinations thereof.

87. The method of claim 83, further comprising adding to said formulation, a pH buffering agent in an amount sufficient to buffer the formulation to a pH in the range of from about 3 to about 7.

88. The method of claim 87, wherein said pH is in the range of from about 4 to about 5.5.

89. The method of claim 87, said pH buffering agent comprises citric acid or a pharmaceutically acceptable salt thereof.

90. The method of claim 83, wherein said medium is selected from the group consisting of purified water, glucose, sterile isotonic saline, physiologically compatible organic solvents and a mixture thereof.

91. A method for treating a disorder in a patient in need thereof, comprising administering to said patient an effective amount of the formulation of claim 1, wherein said disorder is selected from the group consisting of psychosis, agitation, pain, migraine headache, nausea, vomiting, itching, hypertension, benign prostatic hypertrophy, excess gastrointestinal (GI) secretions, and sleeplessness.

92. A method for treating a disorder in a patient in need thereof, comprising administering to said patient an effective amount of the formulation of claim 21, wherein said disorder is selected from the group consisting of psychosis, agitation, pain, migraine headache, nausea, vomiting, itching, hypertension, benign prostatic hypertrophy, excess gastrointestinal (GI) secretions, and sleeplessness.

93. A method for treating a disorder in a patient in need thereof, comprising administering to said patient an effective amount of the formulation of claim 42, wherein said disorder is selected from the group consisting of psychosis, agitation, pain, migraine headache, nausea, vomiting, itching, hypertension, benign prostatic hypertrophy, excess gastrointestinal (GI) secretions, or sleeplessness.
